(19) United States
(12) Patent Application Publication
(10) Pub. No.: US 2009/0093839 A1
(43) Pub. Date: Apr. 9, 2009

(54) Devices and methods for augmenting extragastric banding

(76) Inventor: Brian Kelleher, San Diego, CA (US)

Correspondence Address: Brian Kelleher, 15870 Bemardo Center Drive, San Dieo, CA, 92127, US

(21) Appl. No.: 12/286,974

(22) Filed: Oct. 4, 2008

Related U.S. Application Data
(60) Provisional application No. 60/997,678, filed on Oct. 4, 2007.

(51) Int. Cl. A61M 29/02

(52) U.S. Cl. 606/192

(57) ABSTRACT

The present invention is directed generically to a means for altering the ability of the mammalian body to absorb nutritive content from ingested foodstuffs, and more specifically to an apparatus and method of use for an endolumenal sleeve (referred to also as an “intragastrointestinal device” or “gastrointestinal device”) positioned in the mammalian gastrointestinal (GI) tract. A suitable endolumenal sleeve is comprised of an anchor element and an opening at a proximal end, an elongate lumen or hollow open-ended tube having a transverse dimension, and a distal orifice. Optionally, an exterior aspect of the elongate lumen may include additional modes of attachment to the tissues walls of the GI tract through the use of one or more means for promoting tissue in-growth. The endolumenal sleeve is retained in the GI tract such that a substantial fraction of the food and liquids passing through the GI tract is channeled into the proximal opening and through an interlumenal space defined within the interior space of the endolumenal sleeve. Within the endolumenal sleeve there may be one or more restrictive means to constrain, impede or otherwise control the operative flow of material through the device. An individual restrictive means can either be of a fixed geometry or such means may include one or more elements which are adjustable in nature or function. The elongate lumen of the endolumenal sleeve is formed of a polymer composition suitable for controlled ingress of biological secretions, egress of certain selected nutritional elements, and may comprise either a single tubular structure or a multi-section (i.e. articulated and/or multiple lumen) assembly. When the endolumenal sleeve is in situ within the mammalian gastro-intestinal system, ingested foodstuffs are conveyed from the proximal end to said distal orifice. In typical applications, the proximal end of the endolumenal sleeve is positioned within the physiological region extending from the lower esophagus to the duodenum and the distal orifice is positioned within the physiological region extending from the upper duodenum to the lower jejunum, though further extension into the lower intestine is possible. Through proper selection of position for the endolumenal sleeve proximal and distal ends, combined by selection of the composition used in the fabrication of the elongate lumen, it is possible to finitely control the degree of nutritive absorption performed by the gastrointestinal tract.

### CROSS-REFERENCE TO RELATED APPLICATIONS

This application claims the benefit under 35 U.S.C. 119(e) of U.S. provisional applications Ser. No. 60/997,678 filed Oct. 4, 2007, which is incorporated by reference herein in its entirety.

### STATEMENT REGARDING FEDERALLY SPONSORED RESEARCH

Not Applicable

### BACKGROUND OF THE INVENTION

In the treatment of disorders of the mammalian body, and in particular addressing issues of excessive nutritive consumption thereof, it is known in the medical arts to physically alter the condition within or about a digestive organ so as to effectively change the functionality of that organ within the overall biologic processes of the body. These treatments and physical alterations target organs of interest directed to nutritive absorption within the body and are included within the family of organs generally referred to as the “hollow viscera”, which comprises primarily elements of the gastrointestinal tract.

Surgical modification of the nature or function of hollow viscera to alter nutritive uptake includes numerous procedures ranging from reconstruction of natural tissues to substitution and bypass techniques with natural or artificial implants. One example of gastric reconstruction surgery is the gastric bypass, or Roux-n-Y procedure. In this procedure, the stomach is divided to create a small pouch at the top (in fluid communication with the esophagus) while the main body of the stomach remains connected to the duodenum. A segment of jejunum is then severed, and the distal portion of the severed jejunum is attached to the small stomach pouch, allowing foods and liquids to pass from the esophagus into the pouch and then directly into the jejunum, bypassing the combined body of the stomach, the duodenum and a portion of the jejunum. Secretions from the main body of the stomach continue to flow into the duodenum, as do secretions from the common bile duct. To provide an outlet for these secretions, the proximal end of the severed jejunum is reattached to the small intestine, several feet distal to the point at which it was severed. The efficacy of this procedure comes from three factors. First, as a patient ingests food, the small pouch fills with food quickly, providing at least a partial feeling of fullness after a small meal. The attachment between the pouch and the jejunum is performed by way of a small circular anastomosis, providing a restriction to emptying of the pouch, thereby prolonging the feeling of fullness. Second, a segment of the duodenum and jejunum is bypassed, thereby circumventing a region believe to perform a significant degree of fat absorption. Third, when the portion of the jejunum attached directly to the pouch is exposed to high-calorie and/or high-fat food products, a reaction called “dumping syndrome” occurs, which involves the patient experiencing painful cramping and diarrhea. As a result of these three factors, most gastric bypass patients lose significant weight, partly because consumption is significantly reduced, partly because intake of high-calorie and high-fat foods is negatively reinforced, and partly because of the reduced ability of the patient's body to absorb fat.

Due to the invasive nature of performing gastric surgery, it is recognized by those skilled in the art that there is an ever-present degree of risk to the patient, both directly from the actual procedure as well as in postoperative complications and potentially detrimental side effects. Such risks are of particular concern when the surgery involves resecting one or more organs of the gastrointestinal tract due the greater potential of infiltration of the abdominal cavity by normal gut flora from the bowel, thereby further raising the potential for life-threatening post-operative infection and resulting sepsis. “Gastrointestinal management of the bariatric surgery patient” by Kaplan (Gastroenterol Clin North Am. 2005) and “Gastric bypass revision: lessons learned from 920 cases” by Schwartz et al. (Surgery. 1988) each provides in summary many of the complications encountered in postoperative surgical procedures on patients categorized by body mass index (BMI) as being obese.

Surgical operations are deemed to involve such high risk to the patient that such procedures are considered only as a lifesaving undertaking for morbidly obese individuals, a lifesaving procedure being required at ever increasing regularity. It has been reported by the National Center for Health Statistics (2004) that:
Between 1962 and the year 2000, the number of obese Americans grew from 13% to an alarming 31% of the population.
63% of Americans are overweight with a Body Mass Index (BMI) in excess of 25.0.
31% are obese with a BMI in excess of 30.0.
Childhood obesity in the United States has more than tripled in the past two decades.
According to a then recent U.S. Surgeon General report, obesity is responsible for 300,000 deaths every year.

Beyond statistically significant operative and overall mortality rates, reported complications following the gastric bypass include marginal ulcers and wound infections (Kaplan & Schwartz, ibid.). Common complications include pulmonary emboli, wound infections, gastrointestinal hemorrhage, renal failure, and numerous other disorders. Further, when certain devices are used as a means for effecting digestive uptake (as will be discussed below), additional potentially fatal complications may arise.

To minimize the significant risks of traditional surgery, there has been a growing interest in developing a plurality of endoscopic or less-invasive techniques. One of the more prevalent technologies pursued is the use of inflatable devices inserted into the stomach of the patient to as to occlude gastric volume while minimizing trauma. U.S. Patent Application No. 20080208135 to Annunziata and U.S. Patent Application No. 20080208240 to Paz are representative art citations teaching to placement of inflatable devices for reducing consumption of foodstuffs by a patient through intragastric and interabdominal compression, respectively. In the alternative, medical device technologies have been pursued wherein the operative function is to control the rate an ingested material bolus is allowed to enter the gastrointestinal system. Approaches to controlling the bolus transfer rate include use of intragastric devices and extragastric circumferential constrictions or “gastric banding”. Intragastric devices are represented by U.S. Pat. Nos. 7,118,600 to Dua et al. and 7,146,984 to Stack et al. each of which teaches to means for specifically controlling the rate which ingested material can traverse the esophageal by implanting a valve element in the esophageal sphincter itself. Extragastric “banding” devices are represented by U.S. Pat. Nos. 4,592,339; 4,696,288; and 5,449,368 to Kuzmak et al., U.S. Pat. No. 5,938,669 to Klaiber, et al. and as taught in “Gastric Banding—A new method in the treatment of morbid obesity” by Solhaug (Current Surgery, 1983) each of these references teach to means for specifically controlling the rate at which ingested material can traverse the stomach as a result of external constriction of the gastric cavity; each citation being incorporated herein in their respective entirety. Examples of incrementally more invasive procedures, though still carrying lower risk than open abdominal surgery, is the use of internal joining of the stomach walls, as claimed in U.S. Pat. Nos. 7,037,344 and 7,220,284, both to Kagan, et al.

A further and alternate method for modifying the performance of the GI tract involves the use of a tube or sleeve inserted into the gastrointestinal tract such that ingested material is conveyed from a first point to a second point, resulting in the bypassing of some or all of the biological processing or absorption that would have taken palce along the length of GI tract shielded by the tube or sleeve. U.S. Pat. No. 4,501,264 to Rockey is directed to a device that acts as a barrier layer between the stomach mucosa and the stomach contents. U.S. Pat. No. 5,820,584 to Crabb claims a tube like element extending from the anterior pyloric valve to a position within the small intestine wherein a resilient ring-like proximal anchor is utilized to maintain position of the tube. U.S. Pat. Nos. 7,025,791; 7,267,694 and 7,329,285, each to Levine et al., teach to various bariatric sleeve designs with retention means defined within the gastric cavity. Each of the aforementioned patents listed to Rockey, Crabb and Levine, et al. are incorporated herein in their respective entireties.

The aforementioned ways and means for effecting nutritive uptake by the mammalian gastrointestinal tract have achieved a limited degree of success in treating obese patients. However, there remains an unmet need for an improved bariatric device that is safer, more effective, and is better tolerated by the gastrointestinal mucosa for protracted periods of time.

### SUMMARY OF THE INVENTION

The present invention is directed generically to a means for altering the ability of the mammalian body to absorb nutritive content from ingested foodstuffs, and more specifically to an apparatus and method of use for an endolumenal sleeve (referred to also as an “intragastrointestinal device” or “gastrointestinal device”) positioned in the mammalian gastrointestinal (GI) tract. A suitable endolumenal sleeve is comprised of an anchor element and an opening at a proximal end, an elongate lumen or hollow open-ended tube having a transverse dimension, and a distal orifice. Optionally, an exterior aspect of the elongate lumen may include additional modes of attachment to the tissues walls of the GI tract through the use of one or more means for promoting tissue in-growth. The endolumenal sleeve is retained in the GI tract such that a substantial fraction of the food and liquids passing through the GI tract is channeled into the proximal opening and through an interlumenal space defined within the interior space of the endolumenal sleeve. Within the endolumenal sleeve there may be one or more restrictive means to constrain, impede or otherwise control the operative flow of material through the device. An individual restrictive means can either be of a fixed geometry or such means may include one or more elements which are adjustable in nature or function. The elongate lumen of the endolumenal sleeve may be formed of an impermeable membrane material or a polymer composition suitable for controlled ingress of biological secretions, egress of certain selected nutritional elements, and may comprise either a single tubular structure or a multi-section (i.e. articulated and/or multiple lumen) assembly. When the endolumenal sleeve is in situ within the mammalian gastro-intestinal system, ingested foodstuffs are conveyed from the proximal end to said distal orifice. In typical applications, the proximal end of the endolumenal sleeve is positioned within the physiological region extending from the lower esophagus to the duodenum and the distal orifice is positioned within the physiological region extending from the upper duodenum to the lower jejunum, though further extension into the lower intestine is possible. Through proper selection of position for the endolumenal sleeve proximal and distal ends, combined by selection of the composition used in the fabrication of the elongate lumen, it is possible to alter the flow rate of foods and fluids, as well as the degree of nutritive absorption performed by the gastrointestinal tract.

A preferred embodiment of the present invention includes a proximal anchor element, an elongate lumen, and a distal region wherein the proximal anchor element is comprised of a structure, which is tolerated by the gastrointestinal mucosa for protracted periods of contact. A structure within the proximal anchor element having improved tolerability includes those structures that are supple enough to avoid erosion of the mucosal surface, and which show little resistance to the normal expansion or contraction exhibited by the portion of the GI tract to which the sleeve is attached.

A second embodiment is an endolumenal sleeve in accordance with the present invention which is introduced from the patient esophagus into the gastric cavity and affixed by the proximal anchor element of the endolumenal sleeve to a position between the lower esophageal sphincter and the cardia region of the stomach. The endolumenal sleeve is initially attached to the stomach by the proximal anchor element such that a major fraction of ingested food and liquids conducted through the esophagus is channeled into and through the elongate lumen of the endolumenal sleeve. Optionally one or more restrictive means may be included in the length of elongate lumen bordered on the proximal end thereof by the region of attachment of the anchor region and the distal aspect of the elongate lumen that extends through the pyloric valve. An individual restrictive means can either be of a fixed geometry or such means may include one or more elements which are adjustable in form or function. It is within the purview of the present invention that restrictive means may be a fluid-filled bladder surrounding the elongate lumen. By adjusting the volume of fluid in the bladder (either by endoscopy or via a hydraulic mechanism controlled externally by a wireless link) the amount of constriction or restriction can be varied as needed or desired. In addition, a mechanical assembly powered by a battery source can adjust the restriction means.

A third embodiment of the present invention is an endolumenal sleeve intended for use specifically with patients who have (or will have) a gastric band in place. As described previously, gastric banding involves placing a fixed or adjustable band around the outside of the stomach. Typically, gastric bands are positioned such that the constriction defines a small proximal pouch at the top of the stomach, just below the lower esophageal sphincter. The proximal anchor element of the endolumenal sleeve of this third embodiment is positioned within the pouch, and the elongate lumen extends through the point of constriction. The narrowing created by the gastric band prevents the proximal anchor portion of the endolumenal sleeve from moving into the distal portion of the stomach. The proximal anchor portion is preferably made of a resilient material, and is shaped to be wider than the point of constriction. The elongate lumen is preferably long enough to extend through at least a portion of the duodenum or jejunum.

In a fourth embodiment, the elongate lumen component of the endolumenal sleeve described in each of the previous embodiments further includes a means to reduce the effects of cumulative peristaltic forces acting on the proximal anchoring point of the endolumenal sleeve. Through experimentation, it has been demonstrated by the Applicants (unpublished works) that the force of peristaltic contractions in the bowel surrounding an endolumenal sleeve can act to pull the sleeve away from its point of proximal anchoring. Specifically, when a bolus of food within the sleeve is acted upon by such peristaltic waves, a tugging force is generated in the sleeve that is translated back to the anchoring point. If the endolumenal sleeve is long enough to traverse multiple sites of simultaneous peristaltic contraction, these peristaltic tugging forces can be cumulative, and act to dislodge the sleeve from the proximal anchoring point. There is therefore a need to mitigate these cumulative tugging effects in order to reduce the likelihood of the sleeve becoming dislodged. The features of a preferred endolumenal sleeve aimed at achieving this goal include one or more of the following:
a. The elongate lumen portion of sleeve made to be compliant in response to a local movement (such as by peristaltic contraction) without translating the tugging force back to the proximal anchoring point. Specifically, the sleeve material may have a durometer of composition between 30 A and 100 A, more preferably in the range of 50 A to 80 A, and may be made at least in part from silicone rubber with a wall thickness in the range of 0.003 inches to 0.03 inches;
b. The elongate lumen portion of the sleeve may be comprised of sequential zones of alternating durometer, for example, it may be comprised of longer lengths (e.g., 4 to 40 inches) of material such as expanded PTFE (which are relatively inelastic), joined by shorter lengths (e.g., one-half to 5 inches) of more compliant material (such as described in (a) above) and/or elastic material (such as described in (c) below);
c. The elongate lumen portion of sleeve made to be elastic in response to a local movement (such as by peristaltic contraction) without translating the tugging force back to the proximal anchoring point. Specifically, one or more regions of the elongate tube may be comprised of material having differing degrees of recoverable elongation (elasticity); wherein the sleeve material in a given region may have a degree of elongation recovery of between 10% and 300%, more preferably in the range of 25% and 200% and most preferably in the range of 50% and 100%.
d. The elongate lumen portion of the sleeve has plural attachment points to the gastrointestinal wall wherein the plural attachments points are separated by a distance, whereby that distance is optionally less than or equal to about 10 to 30 inches of elongate lumen length;

Other features and advantages of the present invention will become readily apparent from the following detailed description, the accompanying drawings, and the appended claims.

### BRIEF SUMMARY OF THE FIGURES

The invention will be more easily understood by a detailed explanation of the invention including drawings. Accordingly, drawings which are particularly suited for explaining the inventions are attached herewith; however, it should be understood that such drawings are for descriptive purposes only and as thus are not necessarily to scale beyond the measurements provided. The drawings are briefly described as follows:

FIG. 1 is a cross-sectional view of one embodiment of endolumenal sleeve device shown anchored proximally to the lower esophageal sphincter region, and with a restrictive means positioned between the distal end and the proximal end.

FIG. 2 is a cross-sectional view of an alternate embodiment of endolumenal sleeve device shown anchored to the cardia region of the stomach with an annular ring structure just below the outlet of the esophagus and showing a restrictive means positioned between the distal end and the proximal end.

FIG. 3 is a cross-sectional view of an alternate embodiment of endolumenal sleeve device shown with its proximal opening positioned in the pouch created at the top of the stomach by an adjustable extragastric band apparatus, with the outlet of the endolumenal sleeve extending through the constriction created by the extragastric band and terminating in a distal location in the small intestine.

FIG. 4 is an expanded cross-sectional view of a given region IV-IV from FIG. 1 of elongate lumen wherein a multi-sectioned or articulated elongate lumen assembly is depicted.

FIG. 5 is an expanded cross-sectional view of a given region IV-IV from FIG. 1 of elongate lumen wherein individual transverse attachment points on an exterior aspect of the elongate lumen assembly are depicted.

FIG. 6 is an expanded cross-sectional view of a given region IV-IV from FIG. 1 of elongate lumen wherein continuous attachment points on an exterior aspect of the elongate lumen assembly are depicted.

### DETAILED DESCRIPTION OF THE INVENTION

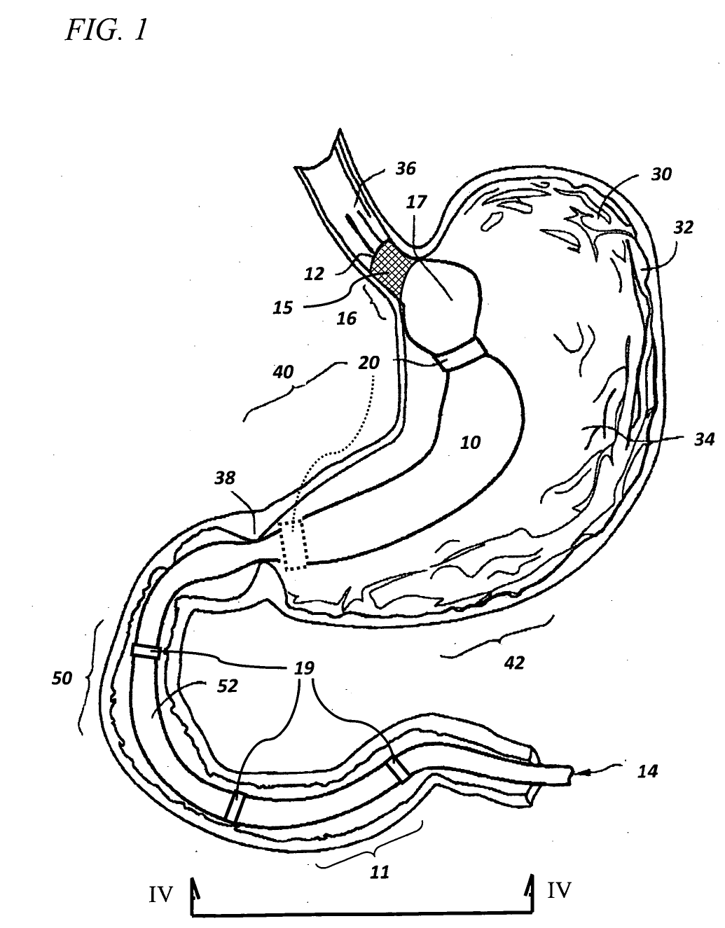

Referring to FIG. 1, there is specifically depicted an endolumenal sleeve 10. Endolumenal sleeve 10 is comprised of primary components: proximal anchor element 15, pouch 17, elongate lumen 52 and distal orifice 14. Proximal anchor element 15 (alternatively referred to as a “proximal anchor point”) includes a proximal opening 12 that is in fluid communication with elongate lumen 52 and acts to form an essentially continuous pathway from proximal opening 12 to distal orifice 14. Optionally, along the length of elongate lumen 52, plural attachments points 19 may be included to further enhance the resistance of elongate lumen 52 to problematic displacement, inversion, eversion and folding caused by peristaltic movement by the gastrointestinal tract. Alternatively, instead of plural attachment points 19, bulbous elements (such as resilient rings, inflatable bladders, foam rings, spring elements and the like) may be incorporated at one or more points along elongate tube 52, particularly in the pyloric canal 37, to anchor tube 52 and prevent it from everting back into the stomach.

The stomach 30 generally has a lumen 34 surrounded by a mucosal tissue and muscle layers 32 and connected to the small intestine 50 by a pylorus valve region 38 and generally exhibits a lesser curvature 40 and a greater curvature 42. The endolumenal sleeve 10 extends through the lumen of the stomach, past duodenum 50 and then terminates at distal orifice 14 in the small intestine. Ingested foodstuffs are conveyed into and through the endolumenal sleeve 10, extending past duodenum 50 (by way of example only) and part of the jejunum 11 of the small intestine, thereby obstructing at least in part the absorption of fats and other related molecules in the duodenum and part of the jejunum.

Further depicted in FIG. 1 is restrictive means 20. Restrictive means 20 acts upon elongate lumen 52 to effectively impede or otherwise control flow through endolumenal sleeve 10. Preferably, restrictive means 20 will be positioned closer to the lower esophageal sphincter, such that pouch 17 will become filled after ingesting about 25-100 cc of food, thereby mimicking the size and location of the pouch created during bariatric surgical procedures such as Roux-n-Y and vertical banded gastroplasty.

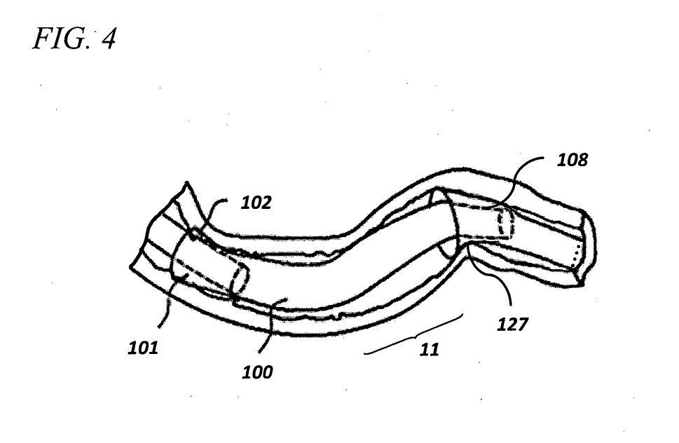

The endolumenal sleeve 10 may be continuous in nature, having a single elongate lumen 52, or may have an interdigitated construction having a plurality of individual elongate lumen fractions combined into a singular articulate elongate lumen form (Reference FIG. 4). The endolumenal sleeve 10 can be fabricated from various solid and/or open or closed cell (i.e. foam) materials such as silicone, ePTFE (W. L. Gore, Flagstaff, Ariz.), cellophane, polyurethane, polyester, polyethylene, and polyamide and combined regions thereof resulting in a variable degree of flexibility. The cross sectional profile of the cavity or hollow defined in the endolumenal sleeve 10 is not a limitation of the present invention, and can include geometries of any suitable application, including but not limited to, linear aspects, curved aspects, multiple lumen, and combinations thereof.

The fabrication of elongate lumen 52 may also include peristaltic-resistant means for reducing the tugging forces exerted on the proximal anchoring point. Peristaltic-resistant means may include incorporation of plural transverse attachments 19 along lumen 52 thereby limiting the chance of peristaltic tugging forces exerted by the bowel on one region of the lumen 52 from affecting regions adjacent to that region. Alternatively, elongate lumen 52 may be made to be compliant in response to a local peristaltic contraction, so lumen 52 will stretch in the region of the local peristaltic force, instead of transmitting the tugging force back into the proximal anchoring point 15. Specifically, the compliant lumen material may have a durometer of composition between 30 A and 100 A, more preferably in the range of 50 A to 80 A, and may be made at least in part from silicone rubber with a wall thickness in the range of 0.003 inches to 0.03 inches. Elongate lumen 52 may, in combination or in the alternative to improved compliance, be made to have an elastic response to a local movement (such as by peristaltic contraction) without translating the tugging force back to the proximal anchoring point 15. Specifically, one or more regions of the elongate lumen 52 may be comprised of material having differing degrees of recoverable elongation (elasticity); wherein the sleeve material in a given region may have a degree of elongation recovery of between 10% and 300%, more preferably in the range of 25% and 200% and most preferably in the range of 50% and 100%.

So as to further improve a counteracting response to peristaltic motion by the gastrointestinal tract, plural regions or sequential zone of alternating durometer and/or elasticity can be employed. The elongate lumen 52 may also utilize regions that not only have a given durometer, but also exhibit recoverable elongation (i.e. elasticity). Suitable materials such as polyurethanes, ABA blocked and silicone rubbers can be employed to provided a desired level of regional elasticity.

Specific to the proximal anchor element 15, additional materials can be formed having a spring or lattice configuration, similar to conventional designs used for esophageal and vascular stenting. Such stent type forms may also be incorporated within a polymer base material to provide an encapsulated or multilayered composite. Initial anchoring or affixing of the proximal anchor element 15 can be achieved by suturing, stapling, clamping, or other mechanical anchors, and utilizing adhesives (e.g. cyanoacrylates, fibrin, methacrylics, ultraviolet-activated).

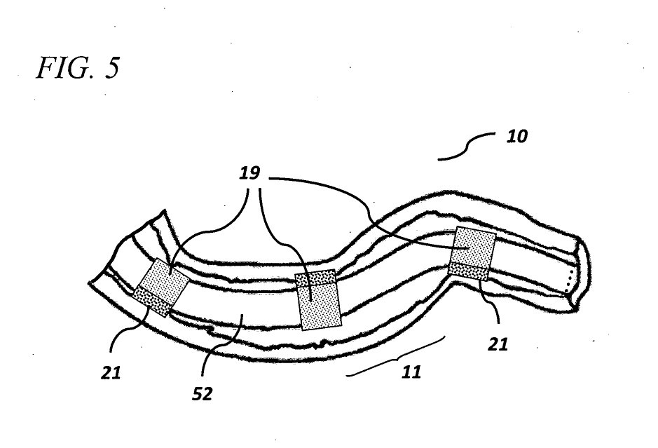

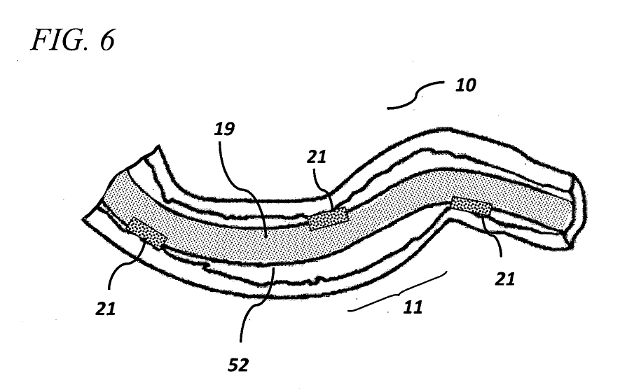

Elongate lumen 52 may include one or more transverse attachment points 19 (Ref, FIGS. 1, 5, and 6). Transverse attachment points 19 are incorporated into an external aspect of the elongate lumen 52 and act as direct attachment points 21 to the gastrointestinal wall. The transverse attachment points 19 are separated by a given distance as measured in the linear direction of the elongate lumen 52, from said proximal opening 12 to said distal orifice 14. The distance between said attachment points along said linear direction of elongate lumen 52 may be spaced in units which are continuous, discontinuous, symmetric, asymmetric, uniform or random, and the combinations thereof. It is preferred that distance between attachment points is optionally less than or equal to about 10 to 30 inches of elongate lumen length.

Endolumenal sleeve 10 can also be manufactured from various materials so as to modify the permeability of the elongate lumen 52 so that the endolumenal sleeve 10 may restrict or enhance the passage of particular substances across the barrier formed by the elongate lumen 52. It is anticipated that the endolumenal bypassing sleeve 10 may made of a non-permeable material that does not allow the passage of biochemical substances, nutrients, liquids or gases to diffuse across the barrier wall formed by the elongate lumen 52. In an alternate construction, elongate lumen 52 is comprised of a semi-permeable membrane that selectively permits certain biochemical substances, nutrients, liquids and gases to diffuse across it. The absolute permeability of the endolumenal sleeve 10 may also be specifically engineered to restrict molecules of a certain molecular weight or size. Further, the endolumenal sleeve 10 may be designed to chemically bond with certain substances so that such substances are not able to pass through and out of elongate lumen 52. An alternate construction includes an endolumenal sleeve 10 which is coated with various pharmacological agents or chemical coatings so as to enhance or inhibit biological or chemical reactions occurring therein or thereupon. It should be noted that such alternate construction may include the interior, exterior, and combinations of same or dissimilar modifications in regions of the interior and/or exterior surface area of the elongate lumen 52.

Varying the permeability of the elongate lumen 52 within endolumenal sleeve 10 may have important medical applications. For example, it may be beneficial where a tubular membrane is affixed to the inside of the small intestine with partial occlusion of the natural lumen and that selectively permits the passage of certain vitamins, amino acids, hormones, biochemical or nutrients that are generally beneficial to the body while blocking the passage of other less-beneficial or otherwise deleterious substances such as fat across the membrane. A selectively active endolumenal sleeve 10 may be of benefit for the treatment of obesity wherein the root cause of the obese condition is malfunction of the normal gastrointestinal tract. Further, the endolumenal sleeve 10 or attached components may be positioned along selective portions of the stomach and small or large intestines as a way to modify the digestion of food by such an afflicted patient.

Restrictive means 20 may consist of a necked-down portion of sleeve 10, or it may be a discrete element such as a resilient doughnut-shaped ring or valve assembly, attached to sleeve 10. The restrictive means 20 may also be of an adjustable design such that it is set to a specific diameter desired by the clinician when implanted in the patient. In addition, the restrictive means 20 can be adjusted to various diameters over time and subsequent to implantation. For example, the restrictive means 20 can be adjusted by inflated with either air or fluid and the inflation port can either be right on the endolumenal sleeve (allowing in situ endoscopic inflation and adjustment) or can be remote. Remote inflation can be done through a removable conduit, which can extend outside the body of the patient. The restrictive means 20 can be inflated with a radiopaque fluid in order to monitor the positioning and degree of inflation using fluoroscopy. The removable conduit(s) can be reversibly connected to restrictive means 20, such as by use of a small needle piercing a self-sealing septum. After the endolumenal sleeve is positioned, and each cuff is inflated, the conduit(s) can be pulled out and removed. Furthermore, the restrictive means 20 can have the capability to communicate with a transmitter/receiver remote means to operate a pump to add or subtract fluid to the bladder, or to operate a mechanical restrictive means (such as a valve operation, irising design) powered with a self-contained power source. The transmitter/receiver remote means can communicate with mechanical assembly utilizing wireless or optical technology (not shown). The mechanical restrictive means can also have the capability to communicate the associated adjusted diameter or bladder pressure back to a remote transmitter/receiver means.

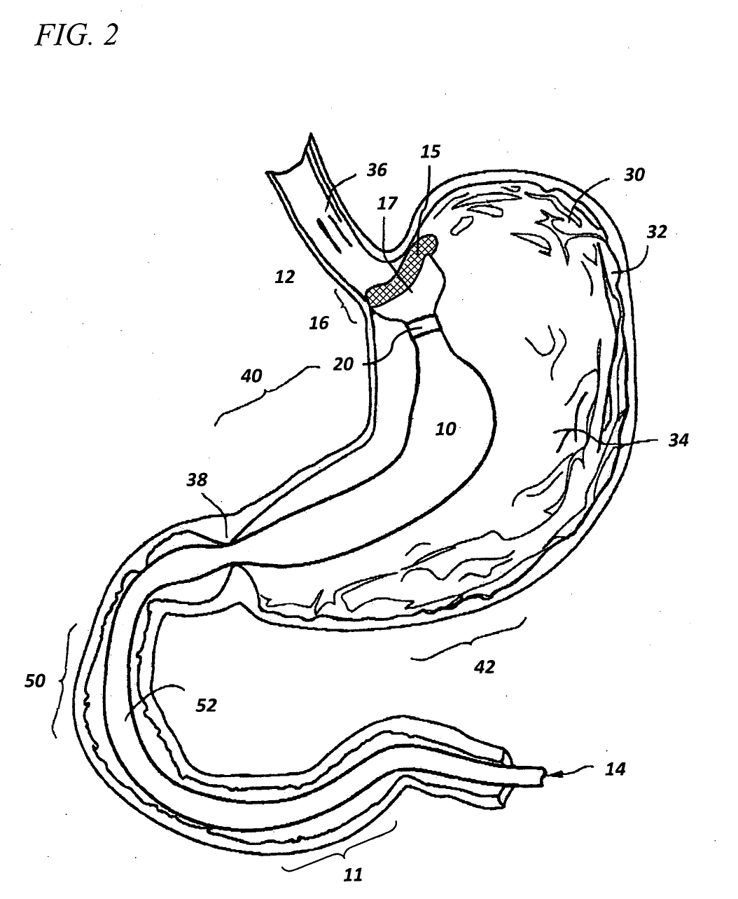

Referring to FIG. 2, proximal anchor element 15 can be affixed near the top of the stomach 30, in the cardia region, just below the lower esophageal sphincter 16. The endolumenal sleeve 10 is attached to the stomach 30 so that a substantial fraction of ingested food coming in to the stomach can be channeled into the proximal opening 12 and subsequently into elongate lumen 52 of endolumenal sleeve 10. Preferably, there is a restriction means 20 in the sleeve, with similar form and function as was described above for the embodiment of FIG. 1.

Proximal anchor 15 preferably has an annular rim or flanged shaped structure to provide a larger surface area for affixing the proximal end 12 of endolumenal sleeve 10 to the cardia region of the stomach. It is known in the art that the attachment of a device to the stomach wall is difficult due to the convulsive nature of the stomach and the sensitivity of the gastric mucosa to erosion and necrosis, as demonstrated by experiments performed by the Applicant (data not provided). As such, one potential solution is to have the proximal anchor 15 at the top of the endolumenal sleeve made from a material such as mesh, fabric, webbing, or netting. One particular example is crystalline polypropylene mesh (under the trade name MARLEX by C. R. Bard) into which stomach wall tissue can grow. One way to achieve this ingrowth is to attach the mesh intimately with the stomach wall such that the mucosa necroses due to the squeezing force of the mesh. Another method for inducing ingrowth is to create a wound along the mucosa prior to attaching the proximal anchor 15 either with mechanical means or freezing, burning or application of chemical agents.

A disadvantage of any gastric restrictive device or surgical procedure is that patients can “cheat” by consuming high-calorie liquids such as soft drinks and milkshakes. To mitigate this, a gastrointestinal endolumenal sleeve which shunts gastric contents past the duodenum to the jejunum (or to whatever point in the small intestine that is desired) can reduce the absorption of these high-calorie liquids, and also potentially trigger “dumping” syndrome.

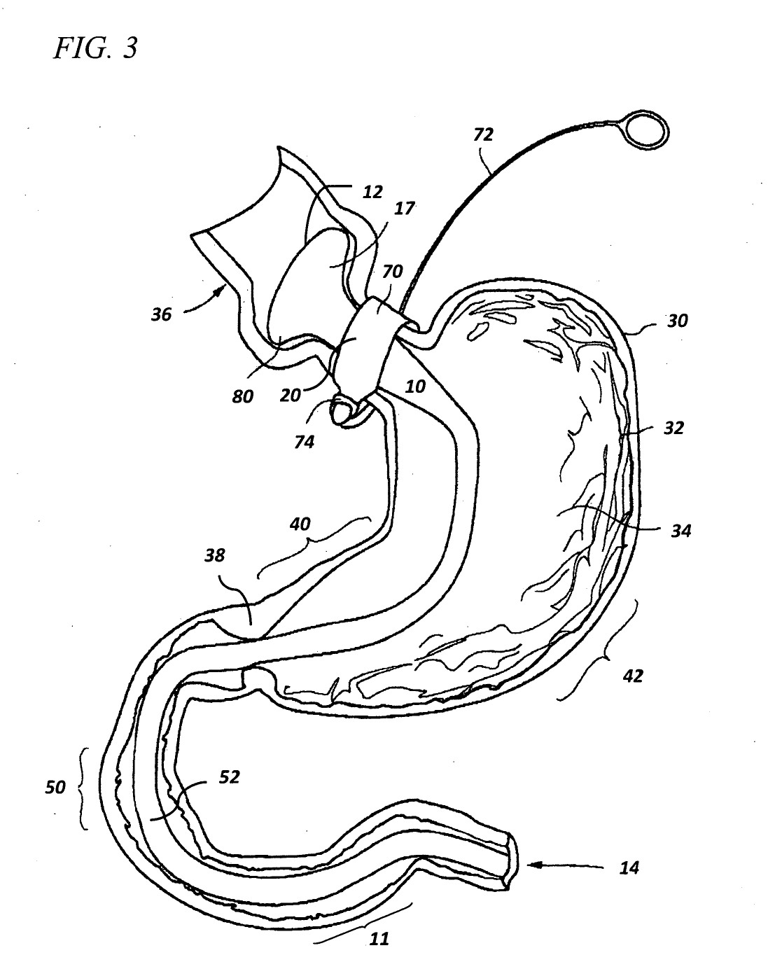

FIG. 3 shows an embodiment of the endolumenal sleeve 10 adapted for use in patients with a gastric band. In this drawing, the gastric band consists of circumferential bladder 70 with clip 74 surrounding a section of the stomach/sleeve and having a fluidic conduit means 72 to adjust the constriction force of the band. The LAP BAND (a registered trademark of Allergan Inc, of Irvine, Calif.) is a representative example of an adjustable gastric band that is wrapped around the outside of the stomach, near the cardia, creating a small gastric pouch at the top of the stomach. The LAP BAND device is adjustable by adding or removing saline from an integral bladder within the band. As with the other conventional gastric restriction procedures, food enters the pouch from the esophagus and fills the pouch. When the rate of food entering the pouch exceeds the rate of food leaving the pouch, the pouch fills up, distending the walls of the stomach and causing a feeling of fullness. Food leaving the pouch falls into the main cavity of the stomach, where digestion occurs normally. It should be noted that while an adjustable extragastric band device is depicted, static or non-adjustable extragastric bands may be employed in the alternative.

As shown in FIG. 3, the proximal opening 12 of sleeve 10 is positioned within pouch 17, and elongate lumen 52 extends through the point of constriction created by band 70. The narrowing created by gastric band 70 prevents the proximal portion of the sleeve from moving into the distal portion of the stomach. The proximal anchor portion is preferably made of a resilient material, and is shaped to be wider than the point of constriction, as shown by the funnel shape region 80 of sleeve 10. Elongate lumen 52 is preferably long enough to extend through at least a portion of the duodenum or jejunum. It is important to note that a significant advantage of this embodiment is that no direct attachment is required between sleeve 10 and the stomach wall.

FIG. 4 depicts an alternate embodiment of endolumenal sleeve 10 wherein the elongate lumen 52 is comprised of plural tube-like, lumen sections 100 interdigitated to form a single continuous tube. As shown, lumen section 100 includes a section proximal end 102 having a section inlet 101. Lumen section 100 tapers down from section proximal end 102 to a smaller diameter section distal end 108. Section distal end 108 is designed to coaxially and securely engage a subsequent section unit 127. This process of interconnecting lumen sections can be repeated until the total length of the desired elongate lumen 52 is achieved.

As can be seen in FIGS. 1 through 6, each device and placement provides important and novel features incremental to that of the prior art, with the additional benefit that such described endolumenal devices can be installed and removed by minimally invasive endoscopic methods, and readily reversed and/or removed when the course of patient treatment is complete.

A final feature of interest being presented herein addresses the danger associated with an endolumenal sleeve prematurely disengaging from the associated anchor and/or attachment points. An endolumenal sleeve which disengages from the respective means of retention becomes poses a significant and immediate risk to the patient as the dislodged device can become a bowel obstruction. The Applicants have devised means for detecting the status and/or mitigating a premature release of an endolumenal sleeve (or other endolumenal therapy device). In practical application, the, there can need to be a fail-safe mechanism to either detect detachment and warn the patient or clinician, or cause the endolumenal sleeve to dissolve or otherwise break into smaller, more innocuous parts or pieces if an in situ device becomes detached. One way of enabling detachment detection is to have a non-structural (not in tension) trigger line connected to the endolumenal sleeve and anchored in the stomach wall. In the event the endolumenal sleeve becomes detached from the associated tissue wall, the trigger line can then pull out or away from a fixture point on the endolumenal sleeve, thereby triggering an embedded micro-transmitter to send a warning signal to a receiver external of the patient. Alternatively, the micro-transmitter may be separately anchored in the tissue wall near the proximal rim of the endolumenal sleeve. A magnet in the endolumenal sleeve can trigger a suitable sensor (i.e. Hall Effect sensor) in the transmitter. If the endolumenal sleeve should disengage from the anchoring/attachment sites, the change in magnetic signal can trigger the sensor and the associated transmitter can emit a warning signal.

An alternate application of the present invention includes use of the endolumenal sleeve as an adjunct to other surgical procedures performed on the mammalian gastro intestinal tract in form a strain relief liner. When, due to other illness, surgery is performed on the gastrointestinal tract, it can be desirable to allow the region of surgery to be provided temporary protection and relief of stress while in a recuperative state. The present invention can be employed as such a strain relief liner which is inserted within a surgically formed area such as an resection, anastomosis, or to shield the securement seam from the negative effects of tension. In particular, this invention relates to strain relief liner devices and methods of placement in hollow organs containing a surgically formed pouch, including gastric restriction procedures in the stomach for treating obesity and gastroesophageal reflux disease (GERD).

Current procedures for forming pouches in hollow organs such as the stomach, intestines and esophagus often employ sutures, staples, clamps, clips or similar attachment means to join together two parts of the organ to form the pouch. These attachment means have a greater chance of remaining secured if the securement elements pass through the thickness of the organ tissue, such as the mucosa of the stomach. It is also critical that the securement seam not be under significant tension or stress. With full thickness of the organ tissue secured arid without tension present at the seam, wall-to-wall healing of the hollow organ pouch, such as that in the stomach, can occur within a significantly reduced period of time (one to two weeks). Until now, there has been no practical device available to reduce or eliminate the amount of tension observed by the attachment means forming the pouch. If tension is present at the securement seam, the securement elements can fail, causing the pouch to become disengaged.

The preferred methods and devices described herein provide for a physical shield for a securement seam to reduce or eliminate tension and stress from the seam itself. Such procedures involve the insertion of a strain relief device placed within a surgically formed pouch of a hollow organ to shield the securement seam from the negative effects of tension. Specific methods and devices described enable a reduction or elimination of tension in the securement seam of a stomach pouch placed for gastric restriction procedures for treating obesity and gastroesophageal reflux disease (GERD).

From the foregoing, it will be observed that numerous modifications and variations can be affected without departing from the true spirit and scope of the novel concept of the present invention. It is to be understood that no limitation with respect to the specific embodiments illustrated herein is intended or should be inferred. The disclosure is intended to cover, by the appended claims, all such modifications as fall within the scope of the claims.

What is claimed:

1. A method for fixating a gastric-bypass device or prosthesis comprising:
fixating in place at least a portion of the device or prosthesis within at least a portion of the gastrointestinal tract lumen;
using at least a portion of a band of material to constrict or restrict the lumen size of said portion of the gastrointestinal tract lumen;
the constriction and/or restriction of said portion of the gastrointestinal tract lumen does not significantly impede the flow of food and/or fluid as it passes through said portion of the gastrointestinal tract lumen;
the constriction and/or restriction of said portion of the gastrointestinal tract lumen will prevent at least a portion of the device or prosthesis from passing through said portion of the gastrointestinal tract lumen; and
the constriction and/or restriction of said portion of the gastrointestinal tract lumen will fixate in place at least a portion of the device or prosthesis within at least a portion of said gastrointestinal tract lumen.

2. A fixation device comprising:
at least a portion of a band of material composed at least in part of collagen fibers that will integrate into or become part of at least a portion of the gastrointestinal tract tissue;
at least a portion of a band of material will cause a constriction or restriction of at least a portion of the gastrointestinal lumen;
the constriction or restriction of at least a portion of the gastrointestinal tract lumen caused by the fixation device will not significantly impede the flow of swallowed food and/or fluid as it passed through said portion of the gastrointestinal tract lumen; and
the constriction or restriction of at least a portion of the gastrointestinal tract lumen caused by the fixation device will cause an intragastrointestinal device or prosthesis to fixate in place within said portion of the gastrointestinal tract lumen.

3. An intragastrointestinal device comprising:
a flanged portion where the greatest dimension of the flanged portion is greater than the greatest dimension of the body portion; the greatest dimension of the flanged portion is greater that the greatest internal dimension of a stricture point within at least a portion of the gastrointestinal tract lumen; and
the intragastrointestinal device may engage or seat on the stricture point such that the intragastrointestinal device is fixated in place.

4. An endolumenal device comprising:
a. a proximal end and a distal end;
b. an elongate lumen having a diameter;
c. a proximal opening near the proximal end of said device, said opening being in fluid communication with the lumen of said elongate lumen; and
wherein said opening is shaped to fit within a gastric pouch created by a gastric banding procedure, and wherein said elongate lumen is sized to extend through the point of constriction in the stomach created by said gastric banding procedure.

5. An endolumenal device as in claim 4, wherein said proximal opening is substantially funnel-shaped, wherein the proximal portion of the funnel shape is of a diameter substantially larger than the diameter of said elongate lumen, and where said funnel-shaped opening joins said elongate lumen near the narrower distal end of the funnel-shaped opening.

6. An endolumenal device as in claim 4 wherein said proximal opening and said elongate tube are made of soft, pliable, biocompatible materials so as to minimize irritation and erosion of adjacent gastric tissue.

7. An endolumenal device as in claim 6 wherein said biocompatible materials may include silicone, ePTFE, cellophane, polyurethane, polyester, polyethylene, and blends thereof.

8. An endolumenal device as in claim 4 wherein said proximal opening channels into said elongate lumen substantially all of the fluids and solids that pass into said gastric pouch.

9. An endolumenal device as in claim 4 wherein said proximal opening channels only a portion of the fluids or solids that pass into said gastric pouch.

10. An endolumenal device as in claim 4 wherein said elongate lumen extends through the pylorus, and whose distal orifice is positioned in one of the duodenum, jejunum or small intestine.

11. An endolumenal device as in claim 10 wherein said elongate tube is anchored at one or more sites distal to the pylorus to prevent said tube from moving backward into the stomach.

12. An endolumenal device as in claim 11 wherein said anchoring comprises the use of one or more bulbous elements that are sized to be too large to move backward through the pylorus.

13. An endolumenal device as in claim 12 wherein said bulbous elements are formed from rings of resilient material, inflatable bladders, foam rings, or spring elements.

14. A method of improving the treatment of obesity in a patient with a gastric band, comprising the steps of inserting an endolumenal channeling device into the patient, said device having a proximal opening which is positioned within the gastric pouch created by the gastric banding procedure, and an elongate lumen, in fluid communication with said proximal opening, which is inserted through the point of constriction created by the gastric banding procedure, said elongate lumen being further positioned to extend through the pylorus and to one of the duodenum, jejunum or small intestine.

15. The method of claim 14 wherein said proximal opening is substantially funnel-shaped and sized such that it cannot move through said point of constriction.

16. The method of claim 14 wherein said elongate lumen is anchored distal to the pylorus in one or more locations, either by attaching the lumen to the tissue wall pushing one or more bulbous elements attached to the lumen through the pylorus.
